(19) United States
(12) Patent Application Publication
(10) Pub. No.: US 2008/0279915 A1
(43) Pub. Date: Nov. 13, 2008

(54) Matrix-Controlled Transdermal Therapeutic System Based on an Adhesive for Administering Norelgestromin or the Combination Thereof with an Estrogen

(76) Inventor: Martina Wilhelm, Holzkirchen (DE)

Correspondence Address: EDWARDS ANGELL PALMER & DODGE LLP, P.O. BOX 55874, BOSTON, MA, 02205, US

(21) Appl. No.: 11/629,046

(22) PCT Filed: Jun. 9, 2005
(86) PCT No.: PCT/EP2005/006213

§ 371 Date: Jul. 8, 2008

(30) Foreign Application Priority Data
Jun. 11, 2004 (DE) 10 2004 028 284.6

(51) Int. Cl. A61K 9/70; A61K 31/57; A61K 31/565; A61P 15/00

(52) U.S. Cl. 424/449; 514/182; 514/170

(57) ABSTRACT

The invention relates to a transdermal therapeutic system comprising an active-ingredient-impermeable top layer, an active-ingredient-containing matrix and a removable protective layer, the matrix comprising or consisting of norelgestromin and an optional oestrogen as active ingredients as well as also a pressure-sensitive hot-melt adhesive and optional auxiliaries.

The invention relates to a matrix-controlled transdermal therapeutic system comprising norelgestromin alone or in combination with an oestrogen, especially ethinyl estradiol. For the matrix, a hot-melt adhesive, especially a styrene block copolymer, can be used.

Norelgestromin (17-deacylnorgestimate; 13-ethyl-17-hydroxy-18,19-dinor-17-alpha-pregn-4-en-20-yn-3-one oxime) belongs to the group of gestagens. Norelgestromin inhibits the release of luteinising hormone (LH) and thus has an ovulation-inhibiting effect. An advantage of 17-deacylnorgestimate is its relatively low androgenic effect in comparison with the prodrug norgestimate or its other metabolites, such as 3-ketonorgestimate and levonorgestrel.

Oestrogens inhibit the secretion of follicle-stimulating hormone (FSH) and thus bring about inhibition of ovulation. Oestrogens include, for example, 17-beta-estradiol and ethinyl estradiol.

Preparations comprising norelgestromin can be used for contraception in women.

Combination preparations comprising norelgestromin and ethinyl estradiol can be used for contraception and for hormone replacement therapy in women.

It is known for matrix-controlled transdermal therapeutic systems (matrix-TTS) to comprise an active-ingredientimpermeable top layer (backing layer), one or more active-ingredient-containing matrix layers and a removable protective layer (release liner). The matrix layer(s) can be self-adhesive or coated with a pressure-sensitive adhesive.

With the aid of a transdermal therapeutic system, the hepatic metabolisation of the active ingredients which occurs in the case of oral administration is by-passed. As a result, the liver is by-passed and gastro-intestinal side effects are avoided. In addition, smaller amounts of active ingredient are sufficient to achieve the same effect. Transdermal administration also has the advantage that the active ingredient, after permeating through the skin, has a direct systemic effect, so that a constant blood plasma level can be ensured. Furthermore, the use of patches that retain their full effectiveness over a period of several days, which are simple and convenient in comparison with oral administration, is a benefit for the patient. Because the system is applied externally, it can fulfil its intended function for a very long period without change.

WO 2004/020189 A1 discloses a transdermal therapeutic system having a top layer (outer layer 2), an intermediate layer (tie layer) and a lower layer (base layer 4), it being possible for the lower layer to be a membrane that controls the release of active ingredient. For the intermediate layer there are proposed inter alia non-pressure-sensitive formulations of styrene block copolymers as thermoplastic elastomers. The known system is said to be suitable for a range of active ingredients, including norelgestromin.

WO 2003/082336 A1 describes a transdermal therapeutic system having a top layer, an active-ingredient-containing adhesive layer and a removable protective layer. The adhesive layer comprises a polyisobutylene/polybutene adhesive and, inter alia, norelgestromin as active ingredient (Example 12).

DE 102 11 832 A1 describes a transdermal therapeutic system in patch form for the controlled delivery of oestrogens and/or gestagens, in which the active-ingredient-containing reservoir comprises a vinylpyrrolidone/vinyl acetate copolymer and cohesion-improving substances, which cohesion improvers may be inter alia styrene/butadiene/styrene block polymers and styrene/isoprene/styrene block polymers.

DE 101 18 282 A1 discloses a pressure-sensitive adhesive for medical uses based on ethylene/vinyl acetate copolymers having a polymer component and an adhesive resin component, wherein the pressure-sensitive adhesive may be a hot-melt pressure-sensitive adhesive (claim 8). An advantage that is emphasised in the case of that known pressure-sensitive adhesive is that the adhesive resin content is relatively low, more specifically in comparison with pressure-sensitive adhesives based on synthetic rubber polymers, such as styrene/isoprene/styrene block copolymers (SIS) or styrene/butadiene/styrene block copolymers (SBS) [0013].

DE 695 06 157 T2 describes a transdermal self-adhesive matrix system for percutaneous administration of a hormone comprising a carrier and a self-adhesive matrix which, in addition to comprising a styrene/isoprene/styrene tri-block copolymer, also comprises an adhesive resin, a plasticiser and crotamiton or an N-substituted 2-pyrrolidone.

Also known from EP 0 836 506 B1 is a transdermal system having a content of norelgestromin alone or in combination with an oestrogen.

It comprises
a) an active-ingredient-impermeable rear layer and
b) an active-ingredient-containing matrix layer comprising polyisobutylene and/or silicone as pressure-sensitive adhesive.

Lactate esters with C12-C18 aliphatic alcohols, oleic acid and polyethylene glycol monolaurate are mentioned as enhancers. Lauryl lactate is preferably used.

U.S. Pat. No. 5,422,119 describes a transdermal therapeutic system for hormone replacement therapy comprising an oestrogen and/or progestin, for example 17-deacetylnorgestimate. Polyacrylates are used as matrix.

A disadvantage of the adhesives described in the literature, such as polyacrylates, polyisobutylenes or silicones, is chiefly their tendency towards cold flow and their low adhesive strength. In addition, the solids content of polymer in the coating solution during production of the matrix in the case of polyacrylates and polyisobutylenes is low (25 to 30%). That is to say, large amounts of solvent have to be used for the production of an active-ingredient-containing matrix, resulting in a substantial impact on the environment.

It is desirable to have a pressure-sensitive adhesive having good adhesive strength and low cold flow. A high adhesive strength ensures that the patch adheres to the skin reliably over the entire duration of use. Low cold flow is desirable in order to prevent the adhesive from oozing out during storage or use of the patch. During storage this results in the patch sticking to the packaging. When the patch is being worn, a high cold flow results in unattractive dirty edges on the skin.

The aim of the invention is to provide a matrix patch comprising norelgestromin and optionally an oestrogen, the matrix patch having good adhesion to the skin and exhibiting low cold flow. A further objective is to provide an environmentally friendly process for the production of the matrix patch.

In accordance with one embodiment, the aim of the invention is achieved by a transdermal therapeutic system comprising an active-ingredient-impermeable top layer, an active-ingredient-containing matrix and a removable protective layer, the matrix comprising or consisting of norelgestromin and an optional oestrogen as active ingredients as well as also a pressure-sensitive hot-melt adhesive and optional auxiliaries.

For hot-melt adhesives, reference can be made to the prior art, for example Van Nostraud Reinhold, Thermoplastic Rubbers: A-B-A Block Copolymers: 217 ff.; Erwins et al., Thermoplastic Rubbers: A-B-A Block Copolymers: 317 ff. In: Satas (Editor), Handbook of Pressure Sensitive Adhesive Technology, 2nd edition, New York 1989, and also EP 0 842 662 A1.

The system according to the invention, especially for contraception, can be provided with an amount of from 100 to 300 μg/day of norelgestromin for a wearing period of from 1 to 10 days.

Furthermore, the system according to the invention can be provided with an amount of about 150 μg/day of norelgestromin for a wearing period of from 1 to 10 days.

Furthermore, the system according to the invention can be provided with an oestrogen selected from the following group: natural 17-beta-estradiol, semi-synthetic estradiol derivative, estradiol ester and 17-alkylated oestrogen.

Furthermore, the system according to the invention can be provided with
11-nitratoestradiol, 7-alpha-methyl-11-nitratoestradiol or 3,17-beta-estradiol dienanthate as semi-synthetic estradiol derivative or
estradiol valerate, estradiol cyprionate, estradiol undecenoate, estradiol decanoate, estradiol benzoate, estradiol succinate or estradiol acetate as estradiol ester or
ethinyl estradiol, ethinyl estradiol 3-isopropyl sulfonate or methyl estradiol as 17-alkylated oestrogen.

Furthermore, the system according to the invention can be provided
with norelgestromin and ethinyl estradiol or
with norelgestromin and 17-beta-estradiol as active ingredients.

Furthermore, the system according to the invention, especially for contraception, can be provided with an amount of from 100 to 300 μg/day of norelgestromin and from 10 to 35 μg/day of ethinyl estradiol for a wearing period of from 1 to 10 days.

Furthermore, the system according to the invention can be provided with an amount of about 150 μg/day of norelgestromin and of about 20 μg/day of ethinyl estradiol for a wearing period of from 1 to 10 days.

Furthermore, the system according to the invention, especially for hormone replacement therapy, can be provided with an amount of from 150 to 350 μg/day of norelgestromin and from 5 to 45 μg/day of ethinyl estradiol for a wearing period of from 1 to 10 days.

Furthermore, the system according to the invention can be provided with an amount of from 175 to 300 μg/day of norelgestromin and from 10 to 35 μg/day of ethinyl estradiol for a wearing period of 7 days.

Furthermore, the system according to the invention, especially for hormone replacement therapy, can be provided with an amount of from 150 to 350 μg/day of norelgestromin and from 20 to 175 μg/day of 17-beta-estradiol for a wearing period of from 1 to 10 days.

Furthermore, the system according to the invention can be provided with an amount of from 175 to 300 μg/day of norelgestromin and from 30 to 150 μg/day of 17-beta-estradiol for a wearing period of 7 days.

According to the invention, the hot-melt adhesive can be
a copolymer of styrene and at least one further monomer selected from the following group: isoprene and/or butadiene and/or ethylene, or
a mixture of such copolymers.

In the system according to the invention,
the copolymer as hot-melt adhesive can be a gradient, block or graft copolymer or
the copolymer mixture as hot-melt adhesive can be a mixture of gradient, block and/or graft copolymers.

The system according to the invention can be provided with a styrene/isoprene/styrene block copolymer (SIS) as copolymer.

Furthermore, the system according to the invention can be provided with a content of penetration enhancer and/or solubiliser and/or filler and/or resin as auxiliary.

Furthermore, the system according to the invention can be provided with a content of penetration enhancer of from 1 to 20% by weight, based on the matrix.

Furthermore, the system according to the invention can be provided with a penetration enhancer selected from the following group:
saturated and/or unsaturated fatty alcohols each having from 8 to 18 carbon atoms and/or esters thereof;
saturated and/or unsaturated fatty acids each having from 8 to 18 carbon atoms and/or esters and/or salts thereof;
polyol fatty acid esters, such as, for example, Cetiol HE;
polyalcohols;
azones;
alkyl methyl sulfoxides;
pyrrolidone;
1-alkylpyrrolidone, such as, for example, N-methyl-2-pyrrolidone;
non-ionic surfactants;
anionic surfactants;
cationic surfactants;
terpenes;
tea tree oil;
saturated and/or unsaturated cyclic ketones;
natural vitamin E and/or synthetic vitamin E and/or vitamin E derivatives;
block copolymers of polyethylene glycol and dimethylsiloxane with a cationic group at one end;
polysiloxanes;
polyoxyethylene-10-stearyl ether and/or a mixture of polyoxyethylene-10-stearyl ether and glyceryl dilaurate;
dodecyl-2-(N,N-dimethylamino)-propanol tetradecanoate and/or dodecyl-2-(N,N-dimethylamino)-propionate;
N-acetyl prolinate esters having more than 8 carbon atoms;
dimethyl-(arylimino)-sulfuran;
a mixture of oleic acid analogues and propylene glycol;
a mixture of padimate O, octyl salicylate, isopropyl myristate, isopropyl palmitate, octyl methoxycinnamate and/or laurocapram;
phospholipids;
polyoxyethylene-7-glycerol monococoate;
2-octyldodecanol;
Transcutol®;
urea; and/or
propylene glycol laurates.

Furthermore, the system according to the invention can be provided with a penetration enhancer selected from the following group:
laurocapram as azone;
DMSO as alkyl methyl sulfoxide;
lauryl ethers, esters of polyoxyethylene, sorbitan fatty acid esters and/or ethoxylated sorbitan fatty acid esters as non-ionic surfactant(s);
sodium lauryl sulfate as anionic surfactant;
cetrimide as cationic surfactant;
lauroglycol as propylene glycol laurate; and/or
lauryl acetate and/or isopropyl myristate.

Furthermore, the system can be provided with a content of soluble polyvinylpyrrolidone as solubiliser.

Furthermore, the system according to the invention can be provided with a content of Kollidon-vinyl acetate as solubiliser.

Furthermore, the system according to the invention can be provided with a content of a filler selected from the following group: silicon dioxide, insoluble polyvinylpyrrolidone, metal oxide, talcum, silicate, stearate, polyethylene, polystyrene and mixtures of a plurality of those fillers.

Furthermore, the system according to the invention can be provided with
titanium oxide and/or zinc oxide as metal oxide and/or
magnesium silicate and/or aluminium silicate as silicate and/or
zinc stearate as stearate.

Furthermore, the system according to the invention can be provided with a content of colophonium resin, phenol resin, alkylphenol resin, petroleum resin and/or xylene resin as resin.

Furthermore, the system according to the invention can be provided with a size of from 10 to 50 cm².

A further embodiment of the invention relates to a process for the production of a transdermal therapeutic system comprising an active-ingredient-impermeable top layer, an active-ingredient-containing matrix and a removable protective layer, the matrix comprising or consisting of norelgestromin and an optional oestrogen as active ingredients as well as also a pressure-sensitive hot-melt adhesive and optional auxiliaries, especially for a system according to the invention described above, wherein
the active ingredient(s) and the hot-melt adhesive are dissolved or suspended in a solvent or solvent mixture,
optionally one or more auxiliaries are added to the resulting solution or suspension;
the resulting solution or suspension, to which one or more auxiliaries may have been added, is applied to a film for the top layer or to a film for the removable protective layer,
the film with the applied solution or suspension is dried,
a film for the removable protective layer is laminated onto the film for the top layer or a film for the top layer is laminated onto the film for the removable protective layer and
one or more transdermal therapeutic systems are stamped out of the resulting laminate.

In the process according to the invention, toluene and/or ethyl acetate and/or heptane and/or ketones, such as methyl ethyl ketone, can be used as solvent.

Furthermore, in the process according to the invention the active ingredient(s), the hot-melt adhesive and the optional auxiliary or auxiliaries can be dissolved or suspended in the solvent or the solvent mixture up to a content of from 40 to 70% by weight (based on the total weight of the resulting solution or suspension).

Furthermore, in the process according to the invention the active ingredient(s), the hot-melt adhesive and the optional auxiliary or auxiliaries can be dissolved or suspended in the solvent or the solvent mixture up to a content of about 50% by weight (based on the total weight of the resulting solution or suspension).

Furthermore, the process according to the invention can be used to produce a patch in accordance with at least one of claims 1 to 25.

Finally, the invention relates to a set comprising three transdermal therapeutic systems according to the invention especially for contraception.

Finally, the invention relates also to a set comprising a plurality of patches according to the invention especially for hormone replacement therapy.

Finally, the invention relates to a set comprising 7-day patches according to the invention.

It has now been found, surprisingly, that hot-melt adhesives are particularly suitable as pressure-sensitive adhesives for a matrix-TTS comprising norelgestromin and optionally an oestrogen, because they exhibit both high adhesive strength and high cohesion and therefore exhibit low cold flow. In addition, the active ingredients are released from a hot-melt adhesive at the level or amount necessary for therapeutic effectiveness (after a certain lag phase) at a rate that is constant over a period of several days. In addition, the impact on the environment in the case of TTS production based on hot-melt adhesives is kept low, because coating solutions with hot-melt adhesives, by virtue of their viscosity properties, can be processed with a high solids content.

For the production of an active-ingredient-containing matrix it is customary for the hot-melt adhesive to be melted at from 80 to 200° C. and mixed with an active ingredient. Norelgestromin and the oestrogens are very temperature-sensitive, however. According to the invention, the active ingredient(s) is (are) therefore dissolved together with a hot-melt adhesive in a solvent. That solvent is then processed further to give the active-ingredient-containing self-adhesive matrix layer.

A matrix patch according to the invention comprises a top layer that is impermeable to the active ingredient(s), an active-ingredient-containing matrix layer and a removable protective layer. The matrix layer comprises norelgestromin and optionally an oestrogen as well as a pressure-sensitive hot-melt adhesive.

Hot-melt adhesives include copolymers with styrene and at least one monomer selected from the group isoprene, butadiene and/or ethylene. Copolymers may be gradient, block or graft copolymers. In addition, the sequence of the monomers may be random or alternating. It is preferable to use block copolymers, especially styrene/isoprene/styrene block copolymers (SIS). Styrene/isoprene/styrene block copolymers are available, for example, under the trade name Califlex D-1111 or Califlex Tr-1107 from Shell Chemical, JSR5000, JSR 5002 or SR5100 from Japan Synthetic Rubber Co. Ltd., or Quintack 3421 from Nippon Zeon Co. Ltd. Styrene block copolymers likewise include Ecomelt M 120 from Collano, Durotak 87-6173 (National Starch) and Dow Corning M6-0153. It is also possible to use mixtures of a plurality of copolymers.

The solids content of the coating solutions according to the invention comprising a hot-melt adhesive can be from 40 to 70%, preferably about 50%. The solids content is understood to mean the amount of polymer (main component), active ingredients and auxiliaries dissolved or suspended in a solvent which gives a viscosity suitable for coating a film for production of the matrix patch.

The matrix patch according to the invention can comprise norgelstromin alone or in combination with an oestrogen.

Oestrogens include natural 17-beta-estradiol, semi-synthetic estradiol derivatives such as 1-nitratoestradiol, 7-alpha-methyl-11-nitratoestradiol, 3,17-beta-estradiol dienanthate, estradiol esters, for example estradiol valerate, cyprionate, undecenoate, decanoate, benzoate, succinate or acetate, as well as 17-alkylated oestrogens such as ethinyl estradiol, ethinyl estradiol 3-isopropylsulfonate or methyl estradiol. Ethinyl estradiol is preferred. A combination of or with norelgestromin and ethinyl estradiol has a beneficial effect on the metabolism, for example they bring about an increase in high-density lipoprotein levels and a reduction in the ratio of low-density lipoprotein to high-density lipoprotein in the serum.

The matrix-TTS according to the invention can be used for contraception in women and for hormone replacement therapy.

For contraception in women, norelgestromin can be used in an amount of from 100 to 300 μper day, preferably about 150 μg/day. For a combination preparation for contraception, preferably from 100 to 300 μper day, especially about 150 μg/day, of norgelgestromin and from 10 to 35 μg/day, especially about 20 μg/day, of ethinyl estradiol are used.

The matrix-TTS according to the invention is designed for a wearing period of from 1 to 10 days, preferably 7 days. The size of the patch can be from 10 to 50 cm².

When the patch is used for contraception, it is applied on the 5th day of the menstrual cycle and replaced as often as is necessary until 21 days have elapsed. In the case of a 7-day patch, 3 patches are accordingly necessary for the period of 21 days.

In the case of hormone replacement therapy for women, norelgestromin can be used in an amount of from 150 to 350 μper day, preferably from 175 to 300 μg/day, together with ethinyl estradiol in an amount of from 5 to 45 μg/day, preferably from 10 to 35 μg/day. For hormone replacement therapy with norelgestromin and 17-beta-estradiol, norelgestromin can be used in an amount of from 150 to 350 μper day, preferably from 175 to 300 μg/day, together with 17-beta-estradiol in an amount of from 20 to 175 μg/day, preferably from 30 to 150 μg/day.

In the case of hormone replacement therapy, a 7-day patch is replaced continuously for the duration of therapy.

The matrix according to the invention, in addition to comprising the active ingredient(s) and one or more hot-melt adhesive(s), may optionally also comprise permeation enhancers, solubilisers, fillers and/or resins.

To increase the penetration of norelgestromin and the oestrogen(s) through the skin it is possible to use permeation enhancers. The following substances are suitable as permeation enhancers:
saturated and/or unsaturated fatty alcohols each having from 8 to 18 carbon atoms and esters thereof;
saturated and/or unsaturated fatty acids each having from 8 to 18 carbon atoms and esters and salts thereof;
polyol fatty acid esters, such as, for example, Cetiol HE;
polyalcohols;
azones (for example laurocapram);
alkyl methyl sulfoxides (for example DMSO);
pyrrolidone;
1-alkylpyrrolidone, for example N-methyl-2-pyrrolidone;
non-ionic surfactants, for example lauryl ethers, esters of polyoxyethylene, sorbitan fatty acid esters, ethoxylated sorbitan fatty acid esters;
anionic surfactants, for example sodium lauryl sulfate;
cationic surfactants, for example cetrimide;
terpenes;
tea tree oil;
saturated and/or unsaturated cyclic ketones;
natural vitamin E (Copherol® F1300); synthetic vitamin E and/or vitamin E derivatives;
block copolymers of polyethylene glycol and dimethylsiloxane with a cationic group at one end;
polysiloxanes;
polyoxyethylene-10-stearyl ether; a mixture of polyoxyethylene-10-stearyl ether and glyceryl dilaurate;
dodecyl-2-(N,N-dimethylamino)-propanol tetradecanoate and/or dodecyl-2-(N,N-dimethylamino)-propionate;
N-acetyl prolinate esters having more than 8 carbon atoms;
dimethyl-(arylimino)-sulfuran;
a mixture of oleic acid analogues and propylene glycol;
a mixture of padimate O, octyl salicylate, isopropyl myristate, isopropyl palmitate, octyl methoxycinnamate, laurocapram;
phospholipids;
polyoxyethylene-7-glycerol monococoate (Cetiol® HE);
2-octyldodecanol (Eutanol® G);
Transcutol®;
urea;
propylene glycol laurates (for example Lauroglycol®).

The permeation enhancers can be used singly or in the form of a mixture of different individual components. The content of permeation enhancers can be from 1 to 20% by weight of the matrix.

The matrix according to the invention can comprise solubilisers, for example soluble polyvinylpyrrolidones such as Kollidon-vinyl acetate, in order to increase the solubility of the active ingredients in the matrix.

As fillers there may be used, for example, silicon dioxide, insoluble polyvinylpyrrolidone, metal oxides such as titanium oxide or zinc oxide, talcum, silicates such as magnesium or aluminium silicate, stearates such as zinc stearate, polyethylene, polystyrene, as well as mixtures thereof.

In order further to increase the adhesive strength, the matrix can additionally comprise resins, for example colophonium resins, phenol resins, alkylphenol resins, petroleum resins and/or xylene resins.

As impermeable top layer there come into consideration films of acetate, acrylate, acrylonitrile/butadiene/styrene, acrylonitrile (methyl methacrylate) copolymers, acrylonitrile copolymers, ethylene ethyl acrylate, ethylene methyl acrylate, ethylene vinyl acetate, ethylene vinyl acetate copolymers, ethylene vinyl alcohol polymers, ionomers, nylon (polyamide), nylon (polyamide) copolymers, polybutylene, polycarbonate, polyester, polyethylene tetraphthalate, thermoplastic polyester copolymers, polyethylene copolymers (high density), polyethylene (high-molecular-weight, high density), polyethylene (intermediate-molecular-weight, high density), polyethylene (linear low density), polyethylene (low density), polyethylene (medium density), polyethylene oxide, polyimide, polypropylene, polypropylene (coated), polypropylene (oriented), polystyrene, polyurethane, polyvinyl acetate, polyvinyl chloride, polyvinylidene chloride and/or styrene/acrylonitrile, which may, if necessary, be metallised or pigmented.

For the removable protective layer there come into consideration polyester, polyethylene, polypropylene, polysiloxane, polyacrylate, ethylene vinyl acetate, polyurethane, polyisobutene or paper, generally coated with silicone and/or polyethylene, or a mixture thereof.

The transdermal therapeutic system or matrix-TTS according to the invention can be packaged in a sachet together with a water-absorber (for example a film of polypropylene with an absorbent). For the packaging of the matrix-TTS it is also possible to use a sachet film, for example of polyethylene/aluminium/paper or paper/polyethylene/aluminium/polyethylene, for example having a thickness greater than 10 μm, with high water-impermeability. In that way it is possible to avoid increasing the water concentration in the matrix, which would promote hydrolysis of the norelgestromin or recrystallisation of the active ingredient.

### Production:

For the production of the matrix-TTS according to the invention, norelgestromin, optionally an oestrogen and at least one hot-melt adhesive are dissolved in a solvent or a solvent mixture. The resulting solution is applied to a film for the removable protective layer and dried. A film for the active-ingredient-impermeable top layer is then applied to that matrix layer.

Suitable solvents for the active ingredients and the hot-melt adhesives are, for example, toluene, ethyl acetate, heptane, ketones or mixtures thereof.

The invention is explained in greater detail by the following Examples, but the scope of the invention is not limited thereby.

### EXAMPLE 1

Composition of a matrix-TTS according to the invention comprising norelgestromin and ethinyl estradiol:

| Ingredients | Content in % by weight |
|---|---|
| Norelgestromin | 8.0 |
| Ethinyl estradiol | 0.5 |
| Ecomelt M120 | 81.5 |
| Isopropyl myristate | 10.0 |

The percentages by weight relate to the matrix.

### Production Process:

Norelgestromin and ethinyl estradiol are dissolved in an n-heptane/ethyl acetate mixture (1:1) (active ingredient solution). The active ingredient solution is then added to the hot-melt adhesive Ecomelt M120 which is dissolved therewith. The amount of n-heptane/ethyl acetate is so chosen that a solids content in the coating composition of 50% is obtained. After addition of isopropyl myristate, the mixture is homogenised, applied to a film for the removable protective layer, for example transparent PET, and dried in a drying tunnel. A PET film (for example Hostaphan RN 19) for the active-ingredient-impermeable top layer is then applied to the resulting matrix. The patches are then stamped. The patches exhibit high adhesive strength and low cold flow.

### EXAMPLE 2

Composition of a matrix-TTS according to the invention comprising norelgestromin and ethinyl estradiol:

| Ingredients | Content in % by weight |
|---|---|
| Norelgestromin | 8.0 |
| Ethinyl estradiol | 0.5 |
| Ecomelt M120 | 83.5 |
| Lauroglycol ® 90 | 8.0 |

The percentages by weight relate to the matrix.

Processing is carried out analogously to Example 1.

1-36. (canceled)

37. A transdermal therapeutic system comprising:
an active-ingredient-impermeable top layer,
an active-ingredient-containing matrix and a removable protective layer,
the matrix comprising norelgestromin.

38. The system of claim 37 wherein the matrix comprises oestrogen.

39. The system of claim 37 wherein the matrix comprises a pressure-sensitive hot-melt adhesive.

40. The system of claim 37 wherein the system comprises from 100 to 300 μg/day of norelgestromin for a wearing period of from 1 to 10 days.

41. The system of claim 37 wherein the system comprises about 150 μg/day of norelgestromin for a wearing period of from 1 to 10 days.

42. The system of claim 37 wherein the system comprises an oestrogen selected from the group consisting of natural 17-beta-estradiol, semi-synthetic estradiol derivative, estradiol ester and 17-alkylated oestrogen.

43. The system of claim 37 wherein the system comprises:
11-nitratoestradiol, 7-alpha-methyl-11-nitratoestradiol or 3,17-beta-estradiol dienanthate as semi-synthetic estradiol derivative;
estradiol valerate, estradiol cyprionate, estradiol undecenoate, estradiol decanoate, estradiol benzoate, estradiol succinate or estradiol acetate as estradiol ester; or
ethinyl estradiol, ethinyl estradiol 3-isoproyl sulfonate or methyl estradiol as 17-alkylated oestrogen.

44. The system of claim 37 wherein the system comprises 1) norelgestromin and ethinyl estradiol or 2) norelgestromin and 17-beta-estradiol.

45. The system of claim 44 wherein the system comprises from 100 to 300 μg/day of norelgestromin and from 10 to 35 μg/day of ethinyl estradiol for a wearing period of from 1 to 10 days.

46. The system of claim 45 wherein the system comprises 150 μg/day of norelgestromin and of about 20 μg/day of ethinyl estradiol for a wearing period of from 1 to 10 days.

47. The system of claim 44 wherein the system comprises from 150 to 350 μg/day of norelgestromin and from 5 to 45 μg/day of ethinyl estradiol for a wearing period of from 1 to 10 days.

48. The system of claim 47 wherein the system comprises from 175 to 300 μg/day of norelgestromin and from 10 to 35 μg/day of ethinyl estradiol for a wearing period of 7 days.

49. The system of claim 44 wherein the system comprises from 150 to 350 μg/day of norelgestromin and from 20 to 175 μg/day of 17-beta-estradiol for a wearing period of from 1 to 10 days.

50. The system of claim 49 wherein the system comprises from 175 to 300 μg/day of norelgestromin and from 30 to 150 μg/day of 17-beta-estradiol for a wearing period of 7 days.

51. The system of claim 37 wherein the hot-melt adhesive comprises 1) a copolymer of styrene and at least one further monomer selected from the group consisting of isoprene, butadiene and ethylene, or 2) a mixture of such copolymers.

52. The system of claim 51 wherein 1) the copolymer as hot-melt adhesive is a gradient, block or graft copolymer or 2) the copolymer mixture as hot-melt adhesive is a mixture of gradient, block and/or graft copolymers.

53. The systems of claim 51 wherein the system comprises 1) a styrene/isoprene/styrene block copolymer (SIS) or 2) a styrene/butadiene/styrene block copolymer (SBS).

54. The system of claim 37 wherein the system comprises one or more auxiliary materials.

55. The system of claim 37 wherein the system comprises one or more penetration enhancers, solubilisers, fillers and/or resins.

56. The system of claim 37 wherein the system has a content of penetration enhancer of from 1 to 20% by weight, based on the matrix.

57. The system of claim 37 wherein the system comprises a penetration enhancer selected from the group consisting of:
saturated and/or unsaturated fatty alcohols each having from 8 to 18 carbon atoms and/or esters thereof;
saturated and/or unsaturated fatty acids each having from 8 to 18 carbon atoms and/or esters and/or salts thereof;
polyol fatty acid esters;
polyalcohols;
azones;
alkyl methyl sulfoxides;
pyrrolidone;
1-alkylpyrrolidone;
non-ionic surfactants;
anionic surfactants;
cationic surfactants;
terpenes;
tea tree oil;
saturated and/or unsaturated cyclic ketones;
natural vitamin E and/or synthetic vitamin E and/or vitamin E derivatives;
block copolymers of polyethylene glycol and dimethylsiloxane with a cationic group at one end;
polysiloxanes;
polyoxyethylene-10-stearyl ether and/or a mixture of polyoxyethylene-10-stearyl ether and glyceryl dilaurate;
dodecyl-2-(N,N-dimethylamino)-propanol tetradecanoate and/or dodecyl-2-(N,N-dimethylamino)-propionate;
N-acetyl prolinate esters having more than 8 carbon atoms;
dimethyl-(arylimino)-sulfuran;
a mixture of oleic acid analogues and propylene glycol;
a mixture of padimate O, octyl salicylate, isopropyl myristate, isopropyl palmitate, octyl methoxycinnamate and/or laurocapram;
phospholipids;
polyoxyethylene-7-glycerol monococoate;
2-octyldodecanol;
Transcutol®;
urea; and
propylene glycol laurates.

58. The system of claim 37 wherein the system comprises a penetration enhancer selected from the group consisting of:
laurocapram as azone;
DMSO as alkyl methyl sulfoxide;
lauryl ethers, esters of polyoxyethylene, sorbitan fatty acid esters and/or ethoxylated sorbitan fatty acid esters as non-ionic surfactant(s);
sodium lauryl sulfate as anionic surfactant;
cetrimide as cationic surfactant;
lauroglycol as propylene glycol laurate; and
lauryl acetate and/or isopropyl myristate.

59. The system of claim 37 wherein the system comprises soluble polyvinylpyrrolidone as solubiliser.

60. The system of claim 37 wherein the system comprises Kollidon-vinyl acetate as solubiliser.

61. The system of claim 37 wherein the system comprises a filler selected from the group consisting of silicon dioxide, insoluble polyvinylpyrrolidone, metal oxide, talcum, silicate, stearate, polyethylene, polystyrene, and mixtures thereof.

62. The system of claim 61 wherein the system comprises:
titanium oxide and/or zinc oxide as metal oxide;
magnesium silicate and/or aluminium silicate as silicate; and/or
zinc stearate as stearate.

63. The system of claim 37 wherein the system comprises colophonium resin, phenol resin, alkylphenol resin, petroleum resin and/or xylene resin.

64. The system of claim 37 wherein the system has a size of from 10 to 50 cm².

65. A process for the production of a transdermal therapeutic system comprising an active-ingredient-impermeable top layer, an active-ingredient-containing matrix and a removable protective layer, the matrix comprising or consisting of norelgestromin and an optional oestrogen as active ingredients as well as also a pressure-sensitive hot-melt adhesive and optional auxiliaries, the process comprising:
dissolving or suspending the active ingredient(s) and the hot-melt adhesive in a solvent or solvent mixture;
applying the resulting solution or suspension to a film for the top layer or to a film for the removable protective layer;
laminating a film for the removable protective layer onto the film for the top layer or laminating a film for the top layer onto the film for the removable protective layer; and
stamping one or more transdermal therapeutic systems out of the resulting laminate.

66. The process of claim 65 wherein toluene and/or ethyl acetate and/or heptane and/or ketones is used as solvent.

67. The process of claim 65 further comprising drying the film with the applied solution or suspension.

68. The process of claim 65 wherein one or more auxiliaries are added to the solution or suspension.

69. The process of claim 65 wherein the active ingredient(s), the hot-melt adhesive and optional auxiliary or auxiliaries are dissolved or suspended in the solvent or the solvent mixture up to a content of from 40 to 70% by weight (based on the total weight of the resulting solution or suspension).

70. The process according to claim 69, wherein the active ingredient(s), the hot-melt adhesive and optional auxiliary or auxiliaries are dissolved or suspended in the solvent or the solvent mixture up to a content of about 50% by weight (based on the total weight of the resulting solution or suspension).

71. A kit comprising a plurality of patches of claim 37 and adapted for hormone replacement therapy.

72. The kit of claim 71 wherein the patches are 7-day patches.

73. The system of claim 37 wherein the system is packaged in a sachet together with a water-adsorber.

74. The system of claim 37 wherein the system is packaged in a sachet film of high water-impermeability, especially of a thickness greater than 10 μm.

75. A method for providing contraception to a subject in need thereof, comprising administering the system of claim 37 to the subject.

76. A method for providing hormone replacement therapy to a subject in need thereof, comprising administering the system of claim 37 to the subject.
